(19) United States
(12) Patent Application Publication
(10) Pub. No.: US 2006/0062850 A1
(43) Pub. Date: Mar. 23, 2006

(54) Controlled release antimicrobial polymer compositions

(76) Inventors: John Chu Chen, Hockessin, DE (US); Julius Uradnisheck, Glen Mills, PA (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/224,618

(22) Filed: Sep. 12, 2005

Related U.S. Application Data
(60) Provisional application No. 60/609,540, filed on Sep. 13, 2004.

(51) Int. Cl. A61K 9/22; A61K 9/14; A61K 33/24

(52) U.S. Cl. 424/468; 424/486; 424/617

(57) ABSTRACT

Antimicrobial polymers that are able to release antimicrobial agents at a controlled rate over a predetermined period of time are provided. The antimicrobial polymers comprise an acid copolymer, an antimicrobial agent, and optionally, an organic acid. The acid copolymer and the organic acid, if present, may be at least partially neutralized. Also provided are methods of manufacturing the antimicrobial polymers of the invention, and articles made therefrom.

### CROSS REFERENCE TO RELATED APPLICATION

The present application claims priority under 35 U.S.C. § 119 to U.S. Provisional Appln. No. 60/609,540, filed on Sep. 13, 2004, which is incorporated herein by reference in its entirety.

### BACKGROUND OF THE INVENTION

1. Field of the Invention

The present invention relates to the field of antimicrobial materials, and, in particular, to polymeric or ionomeric compositions that include antimicrobial agents and that release the antimicrobial agents at a controlled rate over a predetermined period of time. The present invention also relates to methods of synthesizing antimicrobial materials, and to articles that include antimicrobial materials.

2. Description of Related Art

Several patents and publications are cited in this description in order to more fully describe the state of the art to which this invention pertains. The entire disclosure of each of these patents and publications is incorporated by reference herein.

Antimicrobial materials contribute to public safety and health, and reduce economic loss, by killing pathogenic and/or destructive microorganisms or by slowing their multiplication. Therefore, the use of antimicrobial materials has become crucial in medical treatments and devices; odor control of garments or of equipment used in food processing; reduction of pathogen transmission from surfaces shared by many users; and in the preservation of certain materials, such as lumber, food, and the like.

Many antimicrobial agents are known, including antibiotics, that is, natural substances produced by certain groups of microorganisms, and synthetic antibiotics, which are chemically synthesized and identical in molecular structure to the substances produced by microorganisms. Other antimicrobial materials, including natural or non-natural substances that are manufactured chemically, are considered chemotherapeutic agents.

Plastics are commonly used to form useful articles, such as medical devices, because they are relatively low in cost, easily manufactured, and formed with facility into a variety of shapes. Disadvantageously, however, some plastic articles cannot withstand exposure to elevated temperatures and/or high pressure during autoclave sterilization, and may lose their shape upon exposure to these conditions. Thus, medical devices containing such plastics cannot normally be autoclave-sterilized. For these reasons, an inherently antimicrobial polymer that retains the favorable properties of plastic for use in medical devices remains a desirable goal.

A number of polymeric compositions have been reported to possess antimicrobial properties. Some polymers are reported to be inherently antimicrobial. In U.S. Pat. No. 6,316,044, issued to Ottersbach et al., antimicrobial copolymers of t-butylaminoethyl methacrylate and their use as coatings are described.

Polymeric compositions including antimicrobial carboxylates have been reported. For example, U.S. Pat. No. 4,343,788, issued to Mustacich et al., describes elastomeric polymers containing free or releasable carboxylate antimicrobial agents.

Certain polymeric compositions have been rendered antimicrobial by embedded solid particles that include antimicrobial compounds in their bulk or on their surfaces. For example, U.S. Pat. No. 5,180,585, issued to Jacobson et al., describes a polymer that is compounded with particles containing silver, zinc or copper antimicrobial compounds. The particles also have a coating that aids the dispersion and release rate of the antimicrobial compound. In U.S. Pat. No. 5,827,524, issued to Hagiwara et al., the embedded antimicrobial particles have a core of crystalline silica and contain silver, copper or zinc as the antimicrobial agent. U.S. Pat. No. 4,911,898 discloses antimicrobial dispersions of silver containing zeolites in polymers.

Polymeric articles may be rendered antimicrobial by a coating that is antimicrobial or that contains an antimicrobial agent. U.S. Pat. No. 6,582,715, issued to Barry et al., discloses polymeric articles coated with zeolites that are made antimicrobial by treatment with silver and other antimicrobial metal compounds. U.S. Pat. No. 5,770,255, issued to Burrell et al., describes processes of forming antimicrobial coatings containing silver ions on the surface of medical devices.

U.S. Pat. No. 5,843,186, issued to Christ, describes an intraocular lens for the replacement of natural lenses removed in corrective surgery for cataracts. The intraocular lens comprises a conductive organic polymer matrix in which two metals have a chemical half-cell potential difference. When this composite material is exposed to an electrolyte solution, such as saline or body fluids, a current flow results in the release of antimicrobial metal ions into the conductive electrolyte fluid.

Several patents set forth antimicrobial polymers that make use of quaternary ammonium moieties. For example, U.S. Pat. No. 6,194,530, issued to Klesse et al., describes antimicrobial copolymers having quaternary ammonium groups. Likewise, U.S. Pat. No. 6,706,855, issued to Collins et al., describes an antimicrobial polymer, preferably a poly(quaternary ammonium) compound or a polymeric guanide or biguanide, that is covalently bound to a chromophoric marker.

U.S. Pat. No. 6,548,590, issued to Koloski et al., describes a method for making a variety of polymer composites, including an antimicrobial polymer, by evacuating a polymer matrix, such that the free volume of the polymer matrix is rendered available for the infusion of an antimicrobial composite forming material.

In U.S. Pat. No. 6,716,895, issued to Terry, a polymer containing a colloidal metal salt is used as an antimicrobial agent for articles including catheters.

It is apparent from the foregoing that there remains a need for a controlled release antimicrobial polymer that is economically manufactured, that has a release profile that is easily manipulated over relatively long periods of time, and that is easily formed into useful objects, such as catheters.

### SUMMARY OF THE INVENTION

Accordingly, the present invention provides a controlled release antimicrobial polymer composition that is economically manufactured, with a release profile that is easily manipulated over relatively long periods of time, and that is easily molded or formed into useful objects, such as catheters.

The invention also provides melt processible polymer compositions that are inherently antimicrobial, that have a homogeneous morphology, and that are therefore free from the disadvantages of a second dispersed phase in a heterogeneous morphology, such as unpredictably variable mechanical properties and surface roughness or other undesirable aesthetic qualities.

In one aspect, therefore, the invention provides a controlled release antimicrobial polymer composition comprising an E/X/Y acid copolymer or ionomer; at least one organic acid, at least one organic acid salt, or a combination of least one organic acid and at least one organic acid salt; and an antimicrobial agent.

In another aspect, the invention provides a controlled release antimicrobial polymer composition comprising an E/X/Y acid copolymer that is at least partially neutralized with monovalent ions; and an antimicrobial agent.

In another aspect of the invention, a method of making the controlled release antimicrobial polymer composition is provided. The method comprises providing a polymer composition comprising an E/X/Y acid copolymer or ionomer, and incorporating an antimicrobial agent into the polymer composition. The antimicrobial agent may be incorporated into the polymer composition by melt blending or by imbibing, for example.

In another aspect of the invention, a method of imparting antimicrobial properties to a polymer blend is provided. The method comprises providing a polymer blend that includes a polymer composition comprising an E/X/Y acid copolymer or ionomer and, optionally, an organic acid, an additional neutralizing agent, or both, and incorporating an antimicrobial agent into the polymer blend. The antimicrobial agent may be incorporated in to the polymer composition by melt blending or by imbibing, for example.

In another aspect, articles comprising one or more of the controlled release antimicrobial polymer compositions of the invention are provided.

In another aspect of the invention, a method of imparting antimicrobial properties to articles comprising an E/X/Y acid copolymer or ionomer composition, optionally, an organic acid, an additional neutralizing agent, or both is provided. The method comprises imbibing an antimicrobial agent into the article.

### DETAILED DESCRIPTION OF THE INVENTION

The following definitions apply to the terms as used throughout this specification, unless otherwise limited in specific instances.

The term “copolymer” as used herein, alone or in derivative form, e.g., “copolymerize”, refers to polymers containing two or more different monomers. The terms “dipolymer” and “terpolymer” mean copolymers containing only two and three different monomers, respectively.

The term “(meth)acrylic”, as used herein, alone or in derivative form, is shorthand notation for compounds having either acrylic functionality, methacrylic functionality or a mixture comprising compounds of both types, and generally indicates that either or both types are used or can be useful. For example, “alkyl (meth)acrylate” as used herein generically refers to an alkyl acrylate, an alkyl methacrylate, or to a mixture thereof.

The term “ionomer” as used herein, alone or in derivative form, such as “ionomeric”, e.g., refers to an acid copolymer that has been at least partially neutralized with a neutralizing agent such as an inorganic base, and that comprises carboxylate salts with counterions derived from the neutralizing agent.

The term “controlled release”, as used herein, refers to the property of providing a delivery rate that may be constant or variable and that is sustained over a predetermined minimum period of time.

The term “antimicrobial”, as used herein, refers to the property of killing or slowing the growth of microorganisms.

The terms “microorganisms” and “microbes”, as used herein, are synonymous and refer to prokaryotic cells, multicells, animals, and protists; eukaryotic cells, multicells, animals, and protists; viruses; and organisms composed of the above microorganisms in symbiotic associations. The microbes or microorganisms may be pathogenic, opportunistically pathogenic, or benign.

The terms “finite amount” and “finite value”, as used herein, refer to an amount or value that is not equal to zero.

As used herein, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such.

The controlled release antimicrobial polymer composition of the invention comprises one or more acid copolymers. Suitable acid copolymers for use in the present invention are melt processible, or nearly melt processible, and include direct copolymers and graft copolymers.

Direct copolymers are polymerized from a batch that includes all the intended comonomers, and are distinct from graft copolymers, in which a monomer is grafted onto an existing polymer, often in a subsequent free radical reaction.

The acid copolymers suitable for use in the present invention are preferably copolymers of one or more alpha olefins. Suitable alpha olefins include those having from 2 to 6 carbons, and mixtures thereof. Examples of suitable alpha olefins include, without limitation, ethylene, propylene, 1-butene, isobutene, 1-pentene, 2-methyl-1-butene, 3-methyl-1-butene, and isomers of 1-hexene such as 1-hexene and 2-methyl-1-hexene. Ethylene is a particularly preferred alpha olefin.

Suitable acid comonomers for use in the ethylene acid copolymer include C₃ to C₈, α,β-ethylenically unsaturated carboxylic acids. Suitable carboxylic acids include, for example, acrylic acid, methacrylic acid, maleic acid, and maleic acid mono-ester (also referred to in the art as the “half-ester” of maleic acid). Acrylic acid and methacrylic acid are preferred acid comonomers for use in the present invention. One or more acid comonomers may be used to synthesize an acid copolymer.

Other suitable carboxylic acid monomers include but are not limited to: crotonic acid; itaconic acid; fumaric acid; haloacrylic acids such as chloroacrylic acid, for example; citraconic acid; vinylacetic acid; pentenoic acids; alkylacrylic acids; alkylcrotonic acids; alkenoic acids; alkylcrotonic acids; and alkylakanoic acids.

The preferred acid copolymers may optionally contain a third, softening monomer. The term “softening”, as used in this context, refers to a disruption of the crystallinity of the copolymer. Preferred “softening” comonomers include, for example, alkyl (meth)acrylates wherein the alkyl groups have from about 1 to about 8 carbon atoms.

The preferred acid copolymers, when the alpha olefin is ethylene, can thus be described as E/X/Y copolymers, wherein E is ethylene, X is the α,β-ethylenically unsaturated carboxylic acid, and Y is the softening comonomer. X is preferably present at a level of about 0.1 to about 40 wt %, and Y is preferably present at a level of 0 to about 40 wt % of the acid copolymer.

More preferred are acid copolymers in which X is present at a level of about 1 to about 30 wt %, and Y is present at a level of about 0 to about 30 wt % of the acid copolymer. Still more preferably, X is present at a level of about 10 wt % to about 20 wt %.

Acid copolymers suitable for use in the present invention preferably have a weight average molecular weight (M_{w}) greater than about 30 kD, and more preferably greater than about 40 kD.

Examples of acid copolymers suitable for use in the present invention include ethylene/(meth)acrylic acid copolymers. Also included are ethylene/(meth)acrylic acid/n-butyl(meth)acrylate, ethylene/(meth)acrylic acid/iso-butyl(meth)acrylate, ethylene/(meth)acrylic acid/methyl(meth)acrylate, ethylene/(meth)acrylic acid/ethyl(meth)acrylate terpolymers, and the like.

Several preferred acid copolymers for use in the present invention are commercially available. These include Nucrel® acid copolymers, available from E.I. du Pont de Nemours & Co. of Wilmington, Del.

Methods for preparing acid copolymers of ethylene are well known in the art. For example, acid copolymers may be prepared by the method disclosed in U.S. Pat. No. 4,351,931, issued to Armitage. This patent describes acid copolymers of ethylene comprising up to 90 weight percent of ethylene. In addition, U.S. Pat. No. 5,028,674, issued to Hatch et al., discloses improved methods of synthesizing acid copolymers of ethylene when polar comonomers such as (meth)acrylic acid are incorporated into the copolymer, particularly at levels higher than 10 weight percent. Acid copolymers may also be produced by hydrolyzing ethylene acrylate copolymers. U.S. Pat. No. 4,248,990, issued to Pieski, describes the preparation and properties of acid copolymers synthesized at low polymerization temperatures and normal pressures. Other acid copolymers suitable for use in the invention include polymers grafted with carboxylic acid moieties via solution or melt processes, and polymers and copolymers of carboxylic acid containing comonomers made by aqueous dispersion, emulsion or solution polymerization or copolymerization. See, e.g., International Patent Publn. No. WO00/63309, by Capendale et al.

Ethylene acid copolymers with high levels of acid (X) may be difficult to prepare in continuous polymerization reactors due to separation of the monomer and polymer phases. This difficulty can be avoided, however, by the use of co-solvent technology as described in U.S. Pat. No. 5,028,674, or by employing higher reaction pressures than those at which copolymers with lower acid can be prepared.

An acid copolymer suitable for use in the controlled release antimicrobial polymer compositions of the invention may optionally be neutralized to any level that does not result in an intractable copolymer ionomer, i.e., one that is not melt processible, or one that is without useful physical properties. With increasing preference in the order given, about 0.01 mol % to about 100 mol %, about 5 mol % to 100 mol %, about 1 mol % to about 90 mol %, about 5 mol % to about 75 mol %, about 20 mol % to about 60 mol %, or about 30 mol % to about 50 mol % of the acid moieties of the acid copolymer are neutralized by neutralizing agents of one or more compositions. It will be apparent to those of skill in the art that, in acid copolymers having a high acid level, for example more than 15 wt % of acid comonomer, the preferred extent of neutralization, as a percentage of total acid equivalents, is preferably somewhat lower, once more in order to retain melt processibility.

Ionomers suitable for use in the present invention may comprise any feasible counterion or combination of positively charged counterions (cations). Preferred cations of the neutralized acid copolymers may be singly or doubly charged, e.g., monovalent or divalent. When the cations are metal cations, they are preferably selected from among alkali metals (Group 1), alkaline earth metals (Group 2), transition metals (Groups 3 through 12), lanthanides, and actinides. Preferred cations include lithium, sodium, potassium, magnesium, calcium, barium, copper, silver, zinc, mercury, tin, lead, bismuth, cadmium or chromium, ammonium, or a combination of two or more of these cations. More preferably, the cations are monovalent metal cations, such as alkali metal cations. Potassium, silver, and copper are particularly preferred cations for use in the present invention.

Ionomers useful in the practice of the present invention include ionomers obtained from ethylene (meth)acrylic acid (E/(M)AA) dipolymers having a weight average molecular weight (M_{w}) of from about 10 kD to about 500 kD.

Several preferred ionomers for use in the present invention are commercially available. These include Surlyn® ionomers, available from E.I. du Pont de Nemours & Co. of Wilmington, Del., and HIMILAN™ ionomers, available from Mitsui-DuPont Polychemicals Co., Ltd., of Tokyo, Japan.

Methods of preparing ionomers are described in U.S. Pat. No. 3,344,014, for example.

The acid copolymer and/or ionomer may be present in an amount of up to about 100 wt %, based on the total weight of the controlled release antimicrobial polymer composition. In increasing order of preference, the acid copolymer and/or ionomer may be present at a level of at least about 1 wt %, about 10 wt %, about 20 wt %, about 30 wt %, about 40 wt %, or about 50 wt %, based on the total weight of the controlled release antimicrobial polymer composition.

The controlled release antimicrobial polymer compositions of the invention also comprise at least one antimicrobial agent. Suitable antimicrobial agents include any antimicrobial agent that may become associated with the acid copolymer and/or ionomer. Preferably, the antimicrobial agent may become associated with the acid copolymer and/or ionomer via carboxylic acid and/or carboxylate moieties.

The association of the antimicrobial agent with the acid copolymer and/or ionomer may be permanent, reversible, or partially reversible. A permanent association, as by covalent bond formation, e.g., may be preferable when protection of an article itself from colonization is desired. A reversible or partially reversible association may be necessary for the protection of the environs of the article, and may also be necessary to provide controlled release of the antimicrobial agent.

Preferably, the association of the antimicrobial agent with the acid copolymer is reversible under relatively mild conditions, such as physiological conditions, for example. Preferred types of association between the antimicrobial agent and the acid copolymer include, without limitation, hydrogen bonding, complexation, or ionic bonding. Processes to realize those associations include but are not limited to swelling, diffusion with absorption, adsorption, ion exchange interactions, complex formation, ester exchange, acid-base reactions and the like. Acid-base reactions and ion exchange interactions are particularly preferred processes.

Also preferably, the antimicrobial agent is at least partially soluble in water at ambient temperatures and at physiological pH levels. Examples of such antimicrobial agents include water soluble or water miscible alcohols; phenolic compounds such as cresol and phenol; benzoic acid and its salts; sorbic acid and its salts; metal containing compositions; quaternary ammonium compounds; biguanides; bis-biguanide alkanes; short chain alkyl esters of p-hydroxybenzoic acid, commonly known as parabens; N-(4-chlorophenyl)-N′-(3,4-dichlorophenyl) urea, also known as 3,4,4′-trichlorocarbanilide or triclocarban; azoles; chitosan and its derivatives, and derivatives of tetracycline, thienamycin, chloramphenicol, cefoxitin, neomycin, fluoroquinolone, sulfonamides, and aminoglycoside that have hydrophilic solvent or water solubility. Potassium salts of sorbic or benzoic acid are preferred antimicrobial agents.

More preferred antimicrobial agents include water soluble metal containing compositions. Among the suitable metals are included silver, gold, copper, zinc, mercury, tin, lead, bismuth, cadmium, chromium and thallium ions, which have been shown to possess antimicrobial activity. Without wishing to be held to any theory, these antimicrobial metal ions are believed to function by disrupting respiration and electron transport systems upon absorption into cells. Antimicrobial metal ions of silver, gold, copper and zinc are generally considered suitable for in vivo use.

Antimicrobial silver ions are generally considered to be particularly suitable for in vivo use because of their relatively low incidence of undesired side effects at therapeutic concentrations. Moreover, silver ions are efficacious in extremely low concentrations, i.e., “oligodynamic.” Therefore, antimicrobial results can be realized by dissolution of semi-soluble silver salts, by ion exchange of silver cations from substrates with another cation, or by a reaction between water and silver metal, silver oxide, or a sparingly soluble silver salt. Silver salt colloids or silver metal colloids can also provide antimicrobial results due to their relatively large surface to volume ratio, which results in higher reactivity or interchange with the environment compared to bulk materials.

Any metal containing antimicrobial agent that is capable of reacting to associate with a carboxylic acid or carboxylate moiety is suitable for use in the present invention. Examples of suitable metal containing antimicrobial agents include metal salts and organometallic compounds. Also suitable are neutral metal complexes with one or more ligands that are labile with respect to a carboxylic acid or carboxylate moiety, such as diethylenetriamino copper(II) chloride, tetrameric silver amide, silver phosphine dimer, pentahalophenyl silver, and the like.

Although it is believed that, in a preferred embodiment of the invention, antimicrobial metal ions are associated with the polymer through its carboxylic acid or carboxylate moieties, it is contemplated that residual amounts of metal salt starting materials may remain with the controlled release antimicrobial polymer of the invention. Thus, preferred counterions for antimicrobial metal ions include pharmaceutically acceptable counterions such as, without limitation, acetates, benzoates, carbonates, iodates, iodides, lactates, laureates, nitrates, oxides, palmitates, anionic proteins, ionized amino acids, thiosulfates, sulfadiazines, and combinations of two or more thereof. Acetates are more preferred counterions.

Accordingly, when a controlled release antimicrobial polymer composition of the invention is contemplated for use in a medical device for humans, in a urinary catheter, for example, more preferred antimicrobial agents include pharmaceutically acceptable salts of silver, copper, or zinc. Pharmaceutically acceptable silver salts are particularly preferred.

Preferably, the antimicrobial agent is included in the controlled release antimicrobial polymer compositions of the invention in an amount sufficient to provide the desired antimicrobial activity. Those of skill in the art are aware that the amount of an antimicrobial agent that is sufficient to provide the desired antimicrobial activity will vary based on the identity of the antimicrobial agent and on the extent of antimicrobial activity that is desired.

For example, it is to be expected that the amount of antimicrobial agent sufficient to prevent colonization of the surface of an article for several days may be significantly less than the amount required to prevent colonization for several weeks, months, or years. Likewise, the amount of antimicrobial agent sufficient to prevent colonization of the surface of an article of may be significantly less than the amount required to prevent colonization of the article's surface and its environs. It may be desirable for an article to provide antimicrobial activity on its surface and in its environs when the article is a catheter inserted into a bodily orifice, e.g.

Other factors tending to increase or decrease the amount of antimicrobial agent sufficient for use in the present invention will be apparent to those of skill in the art. For example, when the antimicrobial agent is released by ion exchange, high concentrations of countercations in the aqueous phase will increase the rate of exchange with the antimicrobial ion in the invention. Also, high chloride ion concentrations in the aqueous phase might reduce the efficacy of some antimicrobial cations, especially silver. In addition, hydrogen ions or other chemical species in the aqueous phase may synergistically enhance the effect of the antimicrobial metal species of the invention.

General guidance regarding effective amounts of various antimicrobial agents may be found in numerous reference publications, such as the “Physicians' Desk Reference,” 58^{th} Ed., Thomson Healthcare (Montvale, N.J., 2003), for example. Moreover, those of skill in the art, once provided with the teachings herein and the specific Examples of the invention, below, will be well able to conduct the routine experimentation required to determine the effective amounts of various antimicrobial agents in the controlled release antimicrobial polymers of the invention.

In general, however, it is believed that the antimicrobial agent is suitably present in an amount of from about 0.0000001 to about 10 wt %, based on the total weight of the controlled release antimicrobial polymer. It is also believed that, when the antimicrobial agent comprises silver, the silver is suitably present in an amount of from about 0.0001 wt % to about 7.5 wt %; about 0.0005 to about 5 wt %; about 0.001 to about 2 wt %; about 0.005 to about 2 wt %; about 0.005 to about 1 wt %; about 0.01 to about 1 wt %; and about 0.01 to about 0.1 wt %, in increasing order of preference, and based on the total weight of the controlled release antimicrobial polymer.

Controlled release antimicrobial polymer compositions of the present invention may also comprise one or more organic acids. The term “acid”, as used herein with reference to organic acids, e.g., “fatty acid” and “stearic acid”, and unless otherwise limited in specific instances, refers to an acid, a salt of the acid, or a mixture of the acid and one or more of its salts. Thus, an organic acid, as the term is used herein, may have carboxylic acid functionality (—C(O)OH), carboxylate functionality (—C(O)O⁻), or both carboxylic acid and carboxylate functionality.

Suitable organic acids for use in the present invention include nonvolatile, aliphatic organic acids. The suitable organic acids may be saturated or unsaturated. Preferably, the organic acids have from about 6 to about 38 carbon atoms.

Preferred organic acids for use in the present invention include fatty acids, that is, suitable organic acids having from 12 to 36 carbon atoms.

Examples of preferred fatty acids include, without limitation, caproic acid, caprylic acid, capric acid, lauric acid, myristic acid, palmitic acid, stearic acid, hydroxystearic acid, dihydroxystearic acid, oleic acid, linoleic acid, alpha-linolenic acid, dihomo-gamma-linolenic acid, erucic acid, behenic acid, butyric acid, arachidic acid, arachidonic acid, behenic acid, lignoceric acid, cerotic acid, lauroleic acid, myristoleic acid, pentadecanoic acid, palmitoleic acid, margaric acid, ricinoleic acid, elaidic acid, eleostearic acid, licanic acid, eicosenoic acid, eicosapentaenoic acid, docosahexaenoic acid, montanic acid, and isomers thereof. Citric acid is also a preferred organic acid.

Stearic acid, hydroxy- and dihydroxystearic acids, behenic acid, and oleic acid are more preferred fatty acids. Particularly preferred are branched derivatives of fatty acids, including, without limitation, derivatives of oleic acid such as 2-methyl oleic acid, and derivatives of stearic acid such as 2-methyl stearic acid. Also particularly preferred are the salts of organic acids that have branched alkyl substituents or unsaturation and that are non-crystalline at ambient temperatures, including, for example, isostearic acid salts and isooleic acid salts.

When present as salts, the organic acids may be neutralized with any feasible counterion or combination of counterions. Preferably, the counterion includes an alkali metal ion, a transition metal ion, or an alkaline earth metal ion, or a combination of two or more thereof. More preferably, the counterion is selected from potassium, sodium, lithium, magnesium, calcium, barium, gold, copper, silver, zinc, mercury, tin, lead, bismuth, cadmium or chromium ions, or combinations of two or more thereof. Particularly preferred salts include potassium, silver, or copper ions, or a combination of two or more of potassium, silver, or copper ions.

Preferably, the controlled release antimicrobial polymer compositions of the invention include one or more organic acids. The organic acid or acids, when present, are preferably included in a finite amount of at least about 0.1 wt %, at least about 2 weight percent, or at least about 5 wt % of the total weight of the controlled release antimicrobial polymer. Also preferably, the one or more organic acids are present in a finite amount of up to about 10 wt %, 20 wt %, 25 wt %, 30 wt %, 35 wt %, 40 wt %, or 50 wt %, based on the total weight of the controlled release antimicrobial polymer. More preferably, and with increasing preference in the order given, the organic acid or acids are present in an amount of from about 0.1 wt % to about 50 wt %; from about 2 to about 40 wt %; from about 5 to about 35 wt %; from about 5 to about 30%; from about 5 to about 25%; or from about 5 to about 20 wt %.

When an organic acid is present in the controlled release antimicrobial polymer compositions of the invention, the acid is preferably at least partially neutralized. With increasing preference in the order given, about 0.01 mol % to about 100 mol %, about 5 mol % to 100 mol %, about 20 mol % to about 100 mol %, about 30 mol % to about 100 mol %, about 40 mol % to about 100 mol %, or about 50 mol % to about 100 mol % of the acid moieties of the organic acid are neutralized by neutralizing agents of one or more compositions. The suitable and preferred neutralizing agents are as set forth above with respect to ionomers of acid copolymers.

In addition, a controlled release antimicrobial polymer composition of the present invention may optionally comprise one or more additional polymeric components. Suitable additional polymeric components include a second ionomer according to the description above and/or thermoplastic resins, for example. Suitable thermoplastic resins include, without limitation, thermoplastic elastomers, such as polyurethanes; polyetheresters; polyamide ethers; polyether ureas; HYTREL® polyester elastomer, available from E.I. du Pont de Nemours & Co. of Wilmington, Del.; PEBAX™ block copolymers based on polyether-block-amide, available from Atofina Chemicals, Inc., of Philadelphia, Pa.; styrene-butadiene-styrene (SBS) block copolymers; styrene (ethylene-butylene)-styrene block copolymers; polyurethanes; methylcellulose; 4,6-nylon; 6-nylon; polyamides in general (oligomeric and polymeric); polyesters; polyvinyl alcohol; polyolefins including polyethylene, polypropylene, and ethylene/propylene copolymers; metallocene catalized polyolefins, ethylene copolymers with various comonomers, such as ethylene/vinyl acetate, ethylene/(meth)acrylates, ethylene/(meth)acrylic acid, ethylene/epoxy-functionalized monomer, ethylene/CO; metallocene catalized ethylene and its copolymers with, e.g., polyvinyl alcohol or polyacrylate; ethylene/vinyl alcohol copolymers, such as ELVAL™, available from Kuraray Co., Ltd., of Tokyo, Japan; functionalized polymers with grafted maleic anhydride functionality and epoxidized polymers; elastomers, such as ethylene propylene diene monomer (EPDM); metallocene catalyzed polyethylene and its copolymers; ground up powders of the thermoset elastomers; and the like.

Preferably, the additional polymeric component comprises a copolymer of ethylene including, for example, ethylene copolymers with various comonomers, such as ethylene/vinyl acetate, ethylene/(meth)acrylates, ethylene/(meth)acrylic acid and ionomers thereof, ethylene/epoxy-functionalized monomer, ethylene/CO, ethylene/vinyl alcohol, or a blend comprising at least one of these. Also preferably, the additional polymeric component does not include polystyrene. More preferably, the additional polymeric component comprises a polymer selected from the group consisting of: ethylene vinyl acetate (EVA); ethylene/alkyl (meth)acrylate; ethylene/(meth)acrylic acid and ionomers thereof; or a blend comprising at least one of these.

If included, the amount of the optional additional polymeric component may be present in an amount of about 99% by weight to about 1% by weight of the total controlled release antimicrobial polymer composition, provided that it does not destroy the desired antimicrobial properties of the composition of the invention, or of an article comprising the composition of the invention.

The controlled release antimicrobial polymer compositions of the present invention may also include such other additives as are conventional in polymer compositions, for example, antioxidants, UV stabilizers, flame retardants, plasticizers, pigments, processing aids, and the like. Suitable levels of these additives and methods of incorporating these additives into polymer compositions will be known to those of skill in the art. See, e.g., “Modern Plastics Encyclopedia”, McGraw-Hill, New York, N.Y. 1995.

The controlled release antimicrobial polymer compositions of the present invention are expected to control a broad spectrum of microorganisms, including, without limitation, bacteria, yeasts, fungi, mold, algae, viruses, shellfish embryos, and the like.

The present invention also provides methods by which the controlled release antimicrobial polymer compositions described herein may be produced.

In one method of the invention, one or more antimicrobial agents may be physically blended with one or more acid copolymers and/or ionomers. In this method, the suitable acid copolymers, ionomers, antimicrobial agents, and organic acids, the suitable concentration ranges, and the preferred types of association, are as set forth above with respect to the controlled release antimicrobial polymer compositions.

To compound a controlled release antimicrobial polymer composition in this method of the invention, the components of the composition may be blended together in a melt. The melt blend process may be a batch process or a continuous process. The acid copolymer(s) and/or ionomer(s) may first be combined with one another in a “salt and pepper” blend, i.e., a pellet blend of each of the components, or they may be combined with one another via simultaneous or separate metering of the various components, or they may be divided and blended in one or more passes into one or more sections of mixing equipment such as a single screw or twin screw extruder, Banbury, Buss Kneader, Farrell continuous mixer, or other mixing equipment. For example, an extruder with two or more feed zones into which one or more of the ingredients may be added sequentially can be used.

The antimicrobial agent or agents, and the organic acid or acids, if present, may be added to the pellet blend, or they may be combined with the other components of the composition via simultaneous or separate metering addition during the melt blending, or they may be divided and blended in one or more passes into one or more sections of mixing equipment. Alternatively, they may be added to the blend of acid copolymer(s) and/or ionomer(s) after it has been compounded. Preferably, the organic acid and/or acids are preblended with the acid copolymer and/or ionomer in a pellet blend.

Typically, high shear conditions are desirable for efficiently blending the components of a polymer melt. In blending the controlled release antimicrobial polymers of the invention, however, the high processing temperatures that result from the high torque of blending viscous melts may be detrimental. It may therefore be desirable to adjust the mixing rate (e.g., operating speed in rpm of an extruder screw), melt temperature, temperature set point, melt viscosity, and the like, or a combination of two or more processing conditions, to avoid the negative effects of high temperature compounding.

Controlled release antimicrobial polymer compositions that may be affected negatively by high temperature processing include compositions containing certain silver ionomers. When silver salts are compounded with one or more acid copolymers and/or ionomers to form a silver ionomer, or when a silver ionomer is processed with heating, the silver carboxylate moieties will decompose at excessively high temperatures. The decomposition is readily determined visually, as the silver ionomer or the polymer blend containing silver ions will darken, indicating that the Ag⁺ ions have been reduced to elemental silver. It is well known in the art that silver carboxylate will disproportionate to silver metal and carbon dioxide at elevated temperatures and/or upon exposure to ultraviolet irradiation. Some silver carboxylates of the invention show surprisingly high stability, however.

Thus, in general, when the antimicrobial agent in a controlled release antimicrobial polymer composition comprises silver ions, it is preferable to maintain processing temperatures, including melt temperatures for blending, extrusion, molding, and pressing, below the decomposition point of the silver ions. More preferably, and in increasing order of preference, the processing temperatures are kept below 180° C., 175° C., 170° C., 165° C., 160° C., 155° C., 150° C., 145° C., 140° C., 135° C., or 130° C.

In this connection, the change in appearance of a controlled release antimicrobial polymer composition upon decomposition of silver ions is a useful tool for qualitative analysis of these materials. If a controlled release antimicrobial polymer composition prepared by any method has a clear or water white appearance, the presence of silver ions may be ascertained by exposing a sample of the composition to conditions that will result in the decomposition of the silver ions, e.g., UV irradiation. A darkening or the presence of an orange tinge or orange color indicates that silver ions were present before exposure. Likewise, in a sample that exhibits some decomposition before exposure, a darkening or deepening of color indicates that undecomposed silver ions were also present before exposure. Those of skill in the art are well able to determine the molar absorptivity constants, or to construct the calibration curves for sample thickness and silver ion concentration, that would enable the use of this technique for quantitative analysis as well.

Those of skill in the art are also readily capable of determining when processing conditions should be adjusted to avoid or minimize detrimental effects, such as decomposition of the antimicrobial agent. Methods of detecting detrimental effects of unfavorable processing conditions include, e.g., darkening of the polymer blend during compounding.

Higher temperature processing, i.e., up to about 300° C., the temperature at which polymers suitable for use in the invention might begin to decompose, is not expected to be detrimental, however, when the controlled release antimicrobial polymer composition or the antimicrobial agent are unlikely to decompose upon heating. For example, it is expected that potassium benzoate may be compounded with one or more acid copolymers and/or ionomers at any temperature that is reasonably below the decomposition point of the acid copolymer(s) and/or ionomer(s) without negative effect.

When one or more organic acids are present in the controlled release antimicrobial polymers of the invention, they may be added to the polymer blend in the acid form, the salt form, or as a mixture of acid(s) and salt(s). It will be apparent to those of skill in the art that, with the high temperatures and shear rates of extruder processing, or over longer time periods in milder conditions, there will be equilibration, to some extent, between the level of neutralization of the organic acid, and the level of neutralization of the acid copolymer.

Thus, depending on the overall level of neutralization that is desired for the blend, it is possible to over neutralize the acid copolymer, and back titrate by adding the organic acid in its acid form. Conversely, it is possible to add the organic acid, completely neutralized, to an acid copolymer whose level of neutralization is below that which is desired for the polymer blend. Also, the neutralization of the acid copolymer and that of the organic acid can each be adjusted, before blending, to be equal to the desired final level of the controlled release antimicrobial polymer blend. Those of skill in the art recognize that other permutations are possible, and are able to determine which methods may be desirable under particular circumstances.

Those of skill in the art are also aware that a desired balance of cations can be achieved using similar principals and methods. For example, an organic acid in the form of its silver salt may be directly blended with an acid copolymer to produce a controlled release antimicrobial polymer of the invention, provided the organic acid salt of silver is free of impurities that lead to low disproportionation temperatures. Further neutralization, if necessary or desirable, may be provided by adding one or more additional neutralizing agents, such as potassium hydroxide or the like, to the blend. Alternatively, an acid copolymer may be neutralized with a blend of salts of one or more organic acids, the ratio of whose cations corresponds stoichiometrically to the ratio that is desired in the ionomer. Also, an ionomer including one cation may be blended with one or more salts of organic acids that comprise one or more different cations. Over neutralization, if any, may be corrected by back titration with an acid. In these instances, assuming typical melt blending and extruder processing methods are used, it is expected that the concentrations of the cations, which may comprise an antimicrobial agent such as silver ions, will be uniform throughout the bulk of the polymer blend. Again, those of skill in the art recognize that other permutations are possible, and are able to determine which methods of manipulating the cation levels may be desirable under a particular set of circumstances.

In another method of the invention, one or more antimicrobial agents is contacted with an acid copolymer and/or ionomer. As a result, the antimicrobial agent is imbibed into the acid copolymer and/or ionomer to form a controlled release antimicrobial polymer composition of the invention. In this method, the suitable acid copolymers, ionomers, antimicrobial agents, and organic acids, the suitable concentration ranges, and the preferred types of association, are as set forth above with respect to the controlled release antimicrobial polymer compositions, with the proviso that the acid copolymers that are used in this method of the invention are blended with at least one organic acid.

Polymer compositions comprising at least one ionomer are preferred for use in this method of the invention, and polymer compositions comprising at least one ionomer and at least one organic acid are more preferred. Without wishing to be held to any theory, it is believed that carboxylates of the ionomer, and especially of the organic acids, promote the imbibement of antimicrobial agents into ionomers by increasing ion mobility.

In general, preferred antimicrobial agents for this method of incorporation are at least partially soluble in hydrophilic solvents such as water. Silver nitrate is an example of a highly soluble vehicle of the silver cation antimicrobial agent. Without wishing to be held to any theory, it is believed that hydrophilic, non-metallic antimicrobial agents which are to absorb into articles of the invention will normally associate by hydrogen bonding with the carboxylic acid groups or carboxylate anions of the acid copolymer. Silver carboxylate antimicrobial agents and inorganic silver salts or copper salts used in this method of the invention are believed to associate by ionic bonding or by forming a coordination complex with the carboxylate moiety.

Accordingly, the polymer composition is preferably contacted with a hydrophilic liquid comprising the antimicrobial agent(s). Preferably, the liquid contains water, and, more preferably, the liquid is water.

Also preferably, the antimicrobial agent is at least partially dissolved in the liquid, and more preferably, the antimicrobial agent is at least partially dissolved in an aqueous liquid.

In this connection, the term “solution”, as used herein with respect to liquids containing one or more antimicrobial agents, refers to liquids in which at least one solute is an antimicrobial agent, although the soluble antimicrobial agent and other substances, including other antimicrobial agent(s), may also be present in the liquid in both dissolved and undissolved form.

A liquid comprising one or more antimicrobial agents may be contacted with an acid copolymer and/or ionomer by any suitable means, such as, for example, spraying the liquid onto the polymer composition, or at least partially immersing the polymer composition in the liquid. The liquid may be agitated during its contact with the acid copolymer and/or ionomer. If the acid copolymer and/or ionomer is in the form of small particles, the suspension or slurry may be mixed during the contact time.

When the acid copolymer and/or ionomer is contacted by any means with the liquid containing the antimicrobial agent(s), the liquid may preferably be heated. Heating the liquid, provided that no decomposition of the antimicrobial agent(s) or polymer composition results from the raised temperatures, is a suitable means of promoting the diffusion of the antimicrobial agent(s) into the polymer composition.

In a preferred embodiment of this method, a polymer composition comprising an ionomer that is at least partially neutralized by alkali metal ions is contacted with an aqueous solution of one or more metal containing compositions. The polymer composition preferably also comprises at least one organic acid. More preferably, the alkali metal ions comprise potassium, and at least one metal containing composition comprises silver.

In this method of the invention, the concentration of antimicrobial agent within the controlled release antimicrobial polymer compositions may be manipulated by selecting the concentration and temperature of the liquid containing the antimicrobial agent(s), and the duration of the contact between the polymer composition and the liquid.

Preferably, the duration of contact between the polymer composition and the liquid ranges from about 1 minute to about 3 days.

Relatively long durations may be necessary for relatively thick polymer compositions, greater than about 40 mils, to achieve an equilibrium concentration of the antimicrobial agent through the entire thickness of the polymer composition.

The temperature of the liquid containing the antimicrobial agent may range from its melting point to its boiling point, provided that the temperature is not so high as to result in decomposition or unwanted deformation of the polymer composition. It is noted that certain favored polymer compositions for use in the invention are characterized by deformation temperatures as low as 80° C.

Higher concentrations of antimicrobial agent in the liquid generally result in higher concentrations of antimicrobial agent in the controlled release antimicrobial polymer compositions produced by this method of the invention. The concentration of the antimicrobial agent in the liquid may range from 0.1 wt % to about 30 wt %. Lower concentrations may be useful when an antimicrobial agent is very active, or very easily imbibed and released, or when a slow release or low concentration of antimicrobial agent is desired, for example. Conversely, higher concentrations may be useful when a fast release or high concentration of antimicrobial agent is desired, or when the antimicrobial agent is not highly active or not easily imbibed.

Typically, the concentration gradient of the antimicrobial agent with respect to depth within the polymer composition, and therefore the controlled release profile of the antimicrobial agent, may also be manipulated by selecting the concentration of the antimicrobial solution and the duration of the contact between the polymer composition and the antimicrobial solution. For example, higher concentrations of antimicrobial solution and shorter contact times with the polymer composition are expected to result in a rapid initial release of antimicrobial agent, followed by a slower release over a longer period. Alternatively, a slow release of the antimicrobial agent over a long period may be achieved by contacting the polymer composition with a dilute solution of antimicrobial agent for a long time.

In this connection, and without wishing to be held to any theory, it is believed that the mechanism by which the imbibement and release of the antimicrobial agent is achieved includes, without limitation, one or more of swelling, diffusion, complexation, and ion exchange. Accordingly, those of skill in the art are aware that the concentrations and concentration gradients of antimicrobial agents within the controlled release antimicrobial polymers produced by this method of the invention are also affected by other factors that are not explicitly enumerated above and that promote or retard mechanisms such as swelling, diffusion, complexation, and ion exchange. These factors include, without limitation, the chemical composition of the liquid containing the antimicrobial agent, the pH and concentrations of other materials in the liquid, the molecular weight of the acid copolymer or ionomer, the level of acid comonomer in the acid copolymer or ionomer, the level of organic acid in the polymer composition, the extent of neutralization of the polymeric composition, the identity of the cations associated with the polymer composition, and the like.

In another method of the invention, one or more antimicrobial agents are blended with the acid copolymer and/or ionomer melt, and one or more antimicrobial agents, which may be the same or different, are contacted with the controlled release antimicrobial polymer composition.

In this method, the suitable acid copolymers, ionomers, antimicrobial agents, and organic acids, the suitable concentration ranges, the preferred types of association, and the preferred methods of contacting and blending the acid copolymer or ionomer with the antimicrobial agent are as set forth above with respect to the controlled release antimicrobial polymer compositions and the other methods of making the controlled release antimicrobial polymers of the invention.

It is specifically contemplated that this method of the invention may employ more than blending step and/or more than one contacting step, in which the process conditions and/or the antimicrobial agent(s) may be the same or different. Advantageously, this multistep method allows for increased control of the specific efficacy and antimicrobial release profile of the polymer blend, or of an article made from the controlled release antimicrobial polymer composition.

The blending step(s) and the contacting step(s) may be performed in any order that is convenient. Thus, the method may be tailored to the desired end product. It may be desirable, for example, to use a blending procedure to incorporate more robust antimicrobial agents into a controlled release antimicrobial polymer composition, especially if higher temperatures are required, as for melt processing, and afterwards to imbibe any less robust antimicrobial agents that are desired.

Also advantageously, this method of the invention offers the possibility of manipulating the controlled release profile by altering the depth vs. concentration profile of the antimicrobial agent(s) in the bulk polymer. For example, a combination of blending and imbibing would permit the delivery of an imbibed antimicrobial agent over a relatively short initial period, and of a blended antimicrobial agent over a relatively longer period. Alternatively, when the same antimicrobial agent is both blended and imbibed into the acid copolymer and/or ionomer, the delivery rate may be relatively high over a short, initial time period during which both the blended and imbibed portions of the antimicrobial agent are released. The delivery rate may then be relatively lower over a longer time period during which the imbibed portion of the antimicrobial agent is substantially exhausted, and the blended portion of antimicrobial agent is released by diffusing from greater depths within the bulk polymer. Those of skill in the art are able to choose a controlled release profile that is advantageous for the desired device or application.

Also provided by the invention is a method of imparting antimicrobial properties to a polymer blend. In this method, an antimicrobial agent is contacted with a polymer blend comprising an acid copolymer and/or ionomer. The antimicrobial agent becomes associated with the polymer, preferably through interaction with carboxylic acid groups or carboxylate anion groups. The suitable acid copolymers, ionomers, antimicrobial agents, and organic acids, the suitable concentration ranges, the preferred types of association, and the preferred methods of contacting and blending the acid copolymer or ionomer with the antimicrobial agent are as set forth above with respect to the method of making the controlled release antimicrobial polymers of the invention by contacting an acid copolymer and/or ionomer with one or more antimicrobial agents.

The present invention also provides articles comprising a controlled release antimicrobial polymer composition. Any article that may be formed from the controlled release antimicrobial polymer compositions of the invention is specifically contemplated. The immediate products of the above methods of the invention may be in the form of pellets, granules, powders, strands, semi-finished shapes, or any like form. Those of skill in the art are aware that the crude form of the controlled release antimicrobial polymer composition may be dictated by the nature and conditions of the process for making the composition. It is preferable that these crude forms be processed further to produce other articles according to the invention. These articles may be fabricated using well known procedures, including, but not limited to, coating, molding, extruding, spinning, and melt blowing. Medical devices are preferred articles according to the invention, and catheters are more preferred.

In the articles of the invention, the suitable acid copolymers, ionomers, antimicrobial agents, and organic acids, the suitable concentration ranges, the preferred types of association, and the preferred methods of contacting and blending the acid copolymer or ionomer with the antimicrobial agent are as set forth above with respect to the controlled release antimicrobial polymer compositions and the methods of making the compositions of the invention.

A controlled release antimicrobial polymer composition of the invention designed for a specific application is defined in significant part by the molecular weight of the acid copolymer or partially neutralized acid copolymer, the amount of acid comonomer, the amount of softening comonomer, the degree of neutralization of the acid comonomer with cation, and the selection of antimicrobial agent. The selection of these parameters depends on the conversion process to be used to form the finished article from the resin, the mechanical flexibility or softness required in the end-use application, the type of microbiology to be inhibited or controlled, the environment of that microbiology and the duration of the controlling effect on the microbiology.

In general, higher molecular weight resins have higher melt viscosities, and higher degrees of neutralization will generate higher melt viscosities for the partially neutralized acid copolymers. Higher viscosities are valued in extrusion blow molding processes that are used for making large articles or bottles, extrusion of thick articles such as boards, and articles that are thermoformed from wide sheeting such as refrigerator bodies. Higher viscosities are also preferred for articles that must resist high impact or toughness requirements such as auto-body panels and athletic protection equipment.

Lower viscosities, in contrast, are useful for converting resin into thin films, spinning into fibers, molding into parts having fine structures, melt blending with fibrous reinforcements, incorporation with continuous fiber reinforcements, i.e., composites, spray coating followed by fusion, or extrusion coating processes.

Softening comonomers, such as n-butyl acrylate, are used for lowering the modulus of the acid copolymers or partially neutralized acid copolymers. A lower modulus is useful for articles that are to have elastomeric, softness or stretchable attributes, for example catheters, caps, telephone housings, door knobs, or steering wheels. Alternatively, a higher modulus is useful for articles that have structural or weight-bearing requirements, such as stents, flooring, cutting boards, bowls, helmets, toilet bowls, or bath tubs.

The optimal amount of acid comonomer in a controlled release antimicrobial polymer of the invention is dependent on the grease or oil resistance requirement of the article, and on the requirements for stiffness and toughness. Higher levels of acid comonomer result in higher grease resistance, which is a desirable property for articles such as flooring and cutting boards. Higher levels of acid comonomer also result in higher stiffness and toughness, which are desirable properties for articles such as bowls, helmets, steering wheels, hulls, toys, or bicycles. In the present invention, higher acid comonomer content also provides a higher carrying capacity for the antimicrobial agent.

The degree of neutralization of the carboxylic acid groups in the copolymer by cations also affects the melt viscosity of the copolymer. Higher equivalents of cation neutralization produce higher viscosities, which are beneficial to the conversion processes outlined above. Higher neutralization levels, to a limited extent, also improve the toughness of the copolymer.

The type of cation neutralizing species is selected according to the degree of interaction with water or with other hydrophilic liquids. For example, potassium is selected for higher interaction with water; specifically, articles containing potassium counterions imbibe antimicrobial chemicals faster, and release them faster, compared to acid copolymers that contain sodium as a counterion. Articles benefiting from fast release include those that have a temporary immediate use, for example, antiseptic wipes that are activated by moisture. Articles requiring a higher concentration of antimicrobial release, such as those expected to affect microbes within a large volume of stationary or flowing liquid, include toilet bowls, catheters, and porous media or filters.

The criteria for the selection of the type of antimicrobial agent include, without limitation, whether a general or specific microbe is targeted, whether the article is intended for ingestion or internal use, and whether the antimicrobial agents must survive thermal processing of the article. For example, silver cations are antimicrobial agents with a general efficacy; however, care must be taken to limit ingestion of silver cations. Sorbic acid, in contrast, is not as effective on an equal concentration basis as silver cations; however, sorbic acid can be ingested relatively safely at higher concentrations than silver cations.

As noted above, the selection of antimicrobial agent will also depend on the process used to make the shaped article. Many antimicrobial compositions are not thermally stable at temperatures that are typical for melt processing the acid copolymers and ionomers suitable for use in the present invention, i.e., about 120° C. to about 200° C. For example, nisin is a polypeptide antimicrobial agent that will decompose under these melt processing conditions. Advantageously, however, nisin is soluble in water; therefore, nisin may be incorporated into articles of the invention using the liquid contacting methods set forth in detail above.

Silver carboxylate salts also have limited thermal stability. Aliphatic salts are usually not stable at temperatures above about 180° C. Lee, S. J. L., et. al., J. Phys. Chem. B, Vol. 106, No. 11, 2002; and Jamieson, A., et. al., Journal of Polymer Science: Polymer Chemistry Edition, Vol. 16, 2225-2235 (1978). Aromatic salts have higher stability, however, ranging up to 200° C. or higher. Fields, E. K., J. Org. Chem., Vol. 41, No. 6, p. 916 (1976). In general, therefore, the silver carboxylate salt of an acid copolymer also is not expected to be stable above about 180° C. Certain controlled release antimicrobial polymer compositions of the invention, however, are characterized by thermal stability at surprisingly high temperatures.

Further examples of suitable articles comprising the controlled release antimicrobial polymer compositions of the present invention include, without limitation, medical devices, dental devices, food wrap, floor coverings, such as carpets and carpet backings, textile applications such as sportswear, intimate apparel, shoe linings, socks, undergarments, and the like, flooring, cutting boards, bowls, helmets, steering wheels, boat hulls, toys, bicycles, stents, toilet bowls, bath tubs, caps, telephones, cannulae, architectural walls, heating or ventilating duct, kitchen utensils, table tops, food wraps, computer mouse units, mouse pads, swimming pool walls, sterilizing wipes, construction wood, fish nets, conveyor belts used in food processing plants, home or institutional bedding, toothbrushes and other dental equipment, combs and other personal care utensils, door knobs, refrigerators, dish washers, rice cookers, plastic film, vacuum bottles, plastic pails, garbage containers, wall paper, paint, toilet seats, napkins, plastic automobile parts, pens, sand, concrete, breathing masks, porous media, flower pots, hats, shoes, hair setting and styling utensils, watch bands, glasses, purses, umbrella handles, writing utensils, beds, air conditioners and filters, cleaners, humidifiers, sinks, soap dishes, shower curtains, children's chairs, other furniture, refrigerators, can openers, cooking utensils, juicers, caulking agents, calculators, cameras, rental books, checkerboards, credit cards, coatings, and the like.

More specifically, further examples of medical devices include wound closure devices, such as the sutures that are generally described in “Gore-Tex” Suture Bulletins, W. L. Gore & Assoc., Inc. (1986). Examples of devices for purifying or sterilizing aqueous solutions include those that are generally described in Gelman Sciences Process Microfiltration Catalog, (April 1986). Similarly, examples of devices for purifying or sterilizing a gas, such as such as melt blown antimicrobial fibers for sterile filters, include those that are generally described in “Nonwovens in Filtration (1987) Worldwide,” Filter Media Consulting, Inc., (April 1988). Examples of catheters include those that are generally described in “MEDSPEC 1989,” Medical Device Register, Inc., (1989). Examples of suitable devices for storing, transporting or dispensing sterile solutions, devices for controlling odors, wound dressings and garments such as gowns and masks include those that are generally described in “Hospital Supply Index,” Product Analysis, Vol IA and ID, IMS America Ltd., (Third Quarter 1986). Examples of medical implants include those that are generally described in “The Orthopedic Implants and Allied Products Markets Outside the U.S.,” Frost & Sullivan, Inc., (April 1985). Examples of floor coverings, such as carpet backing, include those that are generally described in Edwards, U.S. Pat. No. 3,563,838, Hendersen, U.S. Pat. No. 3,821,062 and Peterson, U.S. Pat. No. 3,502,538. Examples of food wraps include those that are generally described in Chemical Week, Mar. 13, 1983, p. 11. Examples of coatings include those that are generally described in Biomedical Business International, Mar. 2, 1988, pp. 37-38 (Medical), Textil Praxis International, foreign edition with English supplement, 1980, vol. 35, pp. XVI-XXIII (Consumer), and West Marine Products Catalog, (P.O. Box 1020 Watsonville, Calif. 95077) (Summer 1989) pp. 99-100 (Marine). Examples of tests in which the controlled release antimicrobial polymers of the present invention could be used include those that are described in “United States Pharmacopeia, Microbiologial Tests (51).” Antimicrobial Preservative Effectiveness, Vol. XXII pp. 1478-1479 (1990).

Also provided by the present invention are methods of imparting antimicrobial activity to articles, including medical devices, and, in particular, to catheters. According to the methods of the invention, an article comprising an E/X/Y acid copolymer, or an ionomer of an E/X/Y acid copolymer, is contacted with one or more antimicrobial agents. Because, in this method of the invention, the article has already been fabricated, it is preferable to contact the article with a liquid comprising the antimicrobial agent. Consequently, the suitable acid copolymers, ionomers, antimicrobial agents, and organic acids, the suitable concentration ranges, the preferred types of association, and the preferred methods of contacting the acid copolymer or ionomer with the antimicrobial agent are as set forth above with respect to the method of making the controlled release antimicrobial polymer compositions of the invention by contacting an acid copolymer and/or ionomer with one or more antimicrobial agents.

It is specifically contemplated that this method of the invention may employ more than one contacting step, in which the process conditions and/or the antimicrobial agent(s) may be the same or different. Advantageously, this multistep method allows for increased control of the specific efficacy and antimicrobial release profile of an article made from the controlled release antimicrobial polymer composition.

The following examples are provided to describe the invention in further detail. These examples, which set forth a preferred mode presently contemplated for carrying out the invention, are intended to illustrate and not to limit the invention.

### EXAMPLES

Materials and Methods

Unless otherwise noted in specific instances, the following experimental protocols were employed:

Melt compounding on a laboratory scale of antimicrobial agent into formulations of acid copolymer or partially neutralized acid copolymer resins were carried out on a Haake™ Rheocord™ 9000 units, manufactured in Karlsrühe, Germany, and available from the Thermo Electron Corporation of Waltham, Mass. Small batches (typically about 55 g) were made using general purpose mixing rotors running at 50 rpm, 140° C., for a duration of 5 min, and under nitrogen blanketing. The resulting product mass was protected from exposure to light during removal from the processing equipment, and stored in the absence of light.

Product masses, pellets and powders were pressed into films of the specified thicknesses in a hydraulic press manufactured by PHI-Tulip of City of Industry, Calif. The particles were preheated between aluminum plates for one minute at the specified temperature, then subjected to pressure (10,000 to 18,000 psi) at the specified temperature for one minute. The films were removed from the press and cooled to ambient conditions in water.

Samples exposed to UV light were placed in direct contact for one hour with a handheld lamp (8 W, 0.16 amps) available from UVP, Inc. of Upland, Calif., that emitted UV light at a wavelength of 365 nm.

Trace metal concentrations were determined by inductively coupled plasma (ICP) analysis on an apparatus manufactured by PerkinElmer, Inc. of Wellesley, Mass.

In the shaker flask test, duplicate or triplicate test samples were included to determine the variability in testing. The bacteria tested were Klebsiella pneumoniae (ATCC No. 4352) and Staphylococcus aureus (ATCC No. 6538). A dilute suspension of bacteria in 2.5 ml of potassium phosphate buffer (0.6 mM, no chloride) was shaken with a 1.0×1.2 cm sample of film at room temperature for 24 h at 350 rpm in an Orbital shaker, available from VWR International of West Chester, Pa. Bacterial counts were determined by plating on purchased, prepared Trypticase Soy Agar (TSA) plates. The plates were incubated for 24 h at 37° C. Dacron® fibers containing the antimicrobial agent AEM 5700, available from AEGIS Environments of Midland, Mich., were used in the assay as a positive control. The pH of each film and film-bacteria combination was also measured after the 24 h incubation. The level of antimicrobial activity is expressed in terms of colony forming units (“CFU”, e.g., cells) per milliliter as the Δt value,
Δt=log CFU/ml of the Inoculated Control−log CFU/ml of the Test Sample
The antimicrobial polymers were considered effective when the organism count showed a reduction of more than 3 log units, that is, when Δt≧3.0.

Nucrel® acid copolymers are available from E.I. du Pont de Nemours & Co. of Wilmington, Del.

Surlyn® ionomers are available from E.I. du Pont de Nemours & Co. of Wilmington, Del. Surlyn® 8020 is an acid copolymer of ethylene with 10% iso-butylacrylate and 10% methacrylic acid, neutralized to 36% with sodium. Surlyn® 9320 is an acid copolymer of ethylene with 23.5% n-butylacrylate and 10% methacrylic acid, neutralized to 51% with zinc.

Himilan™ ionomers are available from DuPont-Mitsui Polymer Chemicals Co. of Tokyo, Japan. Himilan™ KM-153 includes 11.5 wt % of methacrylic acid comonomer, and is 80% neutralized with potassium ions based on total acid equivalents.

The “8.4% silver ionomer” refers to an ionomer that was produced by mixing an about 10% aqueous dispersion of acid copolymer that was 15% methacrylic acid content with silver nitrate solution. The silver ionomer coagulated and was decanted from the main solution and washed in deionized water. The coagulated mass was dried, reduced to particles, and used as a powder in the experiments described below. The silver content of the ionomer was 8.4% as measured by ICP.

### Example 1

### Processing Temperatures

Surlyn® 8140 (72.57 parts) was melt-blended with isostearic acid (18.14 parts, Century™ 1115, available from Arizona Chemical of Jacksonville, Fla.), and potassium hydroxide (9.29 parts). A portion of the above polymer melt-blend (54 g) was processed by the Haake method with silver acetate (0.0845 g). In a separate experiment, a second portion of the above polymer melt-blend (54 g) was compounded with 0.5 grams of the 8.4% silver ionomer.

Although the set points of the Haake processing were 130° C. and 150° C., respectively, in each run the temperature of the blend rose above 170° C. due to the shear heat created by the high torque required by the high viscosity of the polymer blend melts. The resulting resins were dark colored, indicating that a significant portion of silver cations were converted to metallic silver.

### Example 2

### Compounding Ionomers

Two portions of Surlyn® 1605 were compounded by the Haake method (175° C. set point) with appropriate amounts of the 8.4% silver ionomer to yield two ionomers comprising 0.1 wt % and 0.5 wt % of silver. The actual melt point of both portions rose to 200° C. The bulk composition was dark brown and black for the 0.1% and 0.5% silver containing compositions, respectively.

The above experiment was repeated with a set point of 150° C. The actual measured temperature rose to 175° C. The resulting bulk compositions were flesh colored and light orange for the 0.1% and 0.5% silver containing compositions, respectively.

Each of the two resulting ionomers was pressed into a film of about 4 mil thickness, at 170° C. The 0.1% silver ionomer yielded a film that was tinged with orange, and the 0.5 wt % silver ionomer yielded a flesh colored film. On exposure to UV light, the films shifted to a darker shade of color. When heated (210° C., 2 min), the film including the 0.5 wt % silver ionomer became a much darker orange color. These color changes demonstrate that silver ions were present in the films before exposure to high temperature or UV radiation. The color shift is believed to be due to a conversion of some of the silver cations to silver metal.

In a shake flask test, samples of the 0.1 wt % silver ionomer films, with and without exposure to UV radiation, were efficacious in both phosphate buffer and in water against the gram negative bacterium Klebsiella pneumoniae; however, these films were not efficacious against the gram positive bacterium Staphylococcus aureus in either medium. The 0.5 wt % silver ionomer film, without exposure to UV radiation, was efficacious in both phosphate buffer and water against both organisms. After exposure to UV radiation, the 0.5 wt % silver ionomer film was efficacious in both phosphate buffer and water against K. pneumoniae, but was efficacious against S. aureus only in water.

Without wishing to be held to any theory, it is believed that the antimicrobial activity of the UV-treated films results from an antimicrobial effect of silver metal, which has been reported amply in the literature.

Alternatively, the antimicrobial activity may result from undisproportionated silver cations that remain in the controlled release antimicrobial polymer composition after the UV exposure.

### Example 3

### Compounding Acid Copolymers with Silver Salts

Each of two portions of Nucrel® 3990 (55 g) was compounded with a silver salt (AgNO3, 0.275 g; AgOC(O)CH3, 0.275 g (99.999% pure, obtained from Aldrich Chemical, Inc., of St. Louis, Mo.)) by the Haake method (melt temperature 140° C.). After compounding, the blend with the nitrate salt was white, and the blend with the acetate salt was slightly brown, with an acetic acid odor.

Pellets from each blend were pressed into sheets at a series of temperatures (4 g of pellets; 165° C., 185° C., and 205° C.; 1 min pre-heat and 1 min under pressure to provide 8 mil sheets). The resulting sheets were exposed to UV light, according to the standard protocol.

The sheets made from the silver nitrate blend maintained their white appearance at each processing temperature, with and without UV exposure. These results indicate that the silver is probably not present in the blend as a silver carboxylate, but rather as silver nitrate. It is nevertheless expected that the blend will exhibit antimicrobial properties.

The sheets made from the silver acetate blend exhibited increasing levels of coloration with increasing temperature. When melt-processed into film at 165° C. the color increase from the unprocessed bulk material was small. In addition, each of the sheets darkened further upon exposure to UV radiation. The color shift from the original unexposed samples indicate that the films processed at 205° C. had darkened about 75% as much as they eventually would when exposed to UV radiation. These color changes indicate that the silver is probably present in the blend in silver carboxylate form, as silver acetate and/or silver ionomer. The uniform coloration indicates that the silver is well dispersed throughout the acid copolymer composition, and that the blends can be melt-processed at about 165° C. without unwanted conversion of silver cations to silver metal.

In a shake flask test, the sheets made from the silver acetate blend were found to have significant antimicrobial activity (Δt≧3.0) for both K. pneumoniae and S. aureus in phosphate buffer solution.

### Example 4

### Larger Scale Compounding

The blend compositions listed in Table 1, below, were processed in a 30D Werner & Pfleiderer twin screw extruder operating at 100 rpm with a melt temperature of 140° C. and a flow rate of 10 lb/hr. A one hole die was employed, and the vacuum point was open.

TABLE 1
Blend Compositions

| | | Weight of | | Weight of |
|---|---|---|---|---|
| Sample | | Ionomer, | | Silver |
| No. | Ionomer | lbs | Silver Salt | Salt, g |
| 1 | Nucrel ® 903 | 5 | AgNO₃ | 11 |
| 2 | Nucrel ® 903 | 5 | AgOC(O)CH₃ | 11 |
| 3 | Himilan ™ KM153 | 5 | AgNO₃ | 11 |
| 4 | 50/50 Nucrel ® 903/ | 5 | AgNO₃ | 11 |
| | Himilan ™ KM153 | | | |

Pellets from each blend were pressed into sheets at two temperatures (4 g of pellets; 165° C. and 205° C.; 1 min pre-heat and 1 min under pressure to provide 8 mil sheets). The resulting sheets were exposed to UV light.

The sheets made from the silver nitrate blends with Nucrel® 903 (Sample 1) and 50/50 Nucrel® 903/Himilan™ KM153 (Sample 4) maintained their white appearance at each processing temperature, with and without UV exposure. In contrast, the sheets made from the blend of Himilan™ KM153 with silver nitrate (Sample 3) showed a darkening of color at both processing temperatures, and only slight further darkening after UV irradiation, especially at 205° C. These results indicate that the silver in the NUCREL® blends is probably not bonded to carboxylate groups. The silver in the 100% KM153 sample is probably bonded with carboxylate groups of the KM153. It is nevertheless expected that each of these three blends will exhibit antimicrobial properties.

The sheets made from the silver acetate blend with Nucrel® 903 (Sample 2) maintained their white appearance at both processing temperatures. In addition, each of the sheets darkened more deeply than Samples 1, 3, and 5 upon exposure to UV radiation. The color was uniform throughout the sheet. This color change indicates that the silver is probably present in the blend in a carboxylate form, that it is thermally stable to at least 205° C., and that the silver carboxylate is evenly distributed throughout the sample.

In a shake flask test, the sheets made at both temperatures from Nucrel® 903 (Sample 2) and the sheets made at both temperatures from Himilan™ KM153 (Sample 3) were found to have significant antimicrobial activity (Δt≧3.0) for both K. pneumoniae and S. aureus in phosphate buffer solution. Sheets made from the other blends (Samples 1 and 4) were not tested. It is nevertheless expected that they will exhibit antimicrobial properties.

### Example 5

### Ion Exchange with Silver Salts

Two solutions of silver ions were prepared. Silver nitrate (20 g) was dissolved in distilled water (100 ml) under ambient conditions, as was silver acetate (99% pure, 2 g in 100 ml of distilled water at 80° C.).

Pellets (10 g, approximately 0.13 inch diameter) of Himilan™ KM153 were immersed in one of the silver solutions with hand stirring at ambient temperature for successively increasing periods of time, then rinsed briefly with distilled water. Darkening upon UV exposure verified that silver ions were present in all of the pellets that had been immersed.

Cross-sections of the UV-exposed pellets revealed that the depth of penetration of the silver ions into the pellets also increased with increasing duration of immersion of the resin in a solution of silver ions. After 5.5 h of immersion, silver ions from the acetate solution had penetrated the pellet to a depth of 1.2 mil, and to a depth of 3 mil after 24 h. Silver ions from the nitrate solution had penetrated the pellet to a depth of 0.7 mil after 5.5 h, and to a depth of 2.5 mil after 24 h.

The bulk pellet compositions were analyzed by ICP, and the results of these measurements are summarized in Table 2, below. As expected, the amount of silver in the pellets increased as the time of immersion in the silver solutions increased.

TABLE 2
Silver Ion Concentration vs. Duration of Immersion

| Duration of | μg Ag⁺ per g Resin, | μg Ag⁺ per g Resin, |
|---|---|---|
| Immersion, h | AgOC(O)CH₃ solution | AgNO₃ solution |
| (30 sec) | 405 | 210 |
| 0.1 | 690 | 170 |
| 0.3 | 1750 | 205 |
| 1.5 | 6000 | 470 |
| 5.5 | 10230 | 15885 |
| 24.0 | 40510 | Not reported |

The pellets were pressed into films between release coated aluminum sheets (165° C., with rapid quench in a water cooled press). Samples of the resulting films darkened upon UV exposure, demonstrating that silver carboxylates were present in the films after melt processing.

In a shake flask test, the silver containing films were found to have significant antimicrobial activity (Δt≧3.0) for both K. pneumoniae and S. aureus. The antimicrobial activity of films made from resins that had been immersed in silver ion solution for 30 sec was not tested, however.

### Example 6

### Imbibing Potassium Benzoate

A film of Himilan™ KM153 produced by pressing about 1 g of resin at 150° C. was exposed to a potassium benzoate solution (20 g in 100 ml of distilled water) for one hour with stirring under ambient conditions. The film was immersed in distilled water five times and dried under vacuum overnight. In a shake flask test, this film showed antimicrobial activity against E. Coli (Δt=3.2) in potassium phosphate buffer solution (0.6 mM, no chloride). A control film of Himilan™ KM153 that was prepared according to the same protocol but not imbibed with potassium benzoate did not exhibit antimicrobial activity, although, upon immersion, both films raised the alkalinity of the bacterial broth to 9.1 to 9.6 pH units.

### Example 7

### Potassium Ionomers and Potassium Fatty Acid Salts Effect on Imbibing Process

A sheet approximately 50 mil thick was produced from pellets of each of Himilan™ KM153, Surlyn® 8020, and Surlyn® 9320, by the standard methods outlined above. Four rectangles, each 1 in by 0.2 in, were cut from each of the three sheets. Similarly, four rectangles, 50 mil thick and measuring 1 in by 0.2 in, were generated from the composition of Example 1, before the application of silver.

The four rectangles of each composition were placed in plastic bags containing a solution of silver nitrate (20% aqueous, 10 g). Rectangles were removed from the solution at predetermined time intervals (1 h, 5-6 h, 30 h and 96 h). The rectangles were washed for about 1 minute under running distilled water, dried in vacuo overnight at about 70° C., and then exposed to UV radiation. The color of each rectangle was recorded, and the rectangles were analyzed for silver by ICP.

TABLE 3
Qualitative and Quantitative Analysis for Sllver

| Sheet | Color for 6 h | Color for 92 h | ppm silver | ppm silver |
|---|---|---|---|---|
| Sample | exposure | exposure | 6 h | 92 h |
| KM153 | orange | dark orange | 300 | 1300 |
| 8020 | light orange | light orange | Not | Not |
| | | | reported | reported |
| 9320 | white | white | Not | Not |
| | | | reported | reported |
| Example 1 | dark orange | brown | 2200 | 5000 |

These results show that potassium counterions and, to a greater extent, potassium fatty acid salts increase the rate and extent of the absorption of silver antimicrobial agent into the acid copolymer or ionomer to yield the controlled release antimicrobial polymer compositions of the invention.

### Examples 8 and 9

### Film on Lawn Assay

Two films were prepared by the extrusion casting method. The compositions and gauges of the films are set forth in Table 4, below.

TABLE 4
Film Compositions

Compositions (wt %)

| | Example 8 | Example 9 |
|---|---|---|
| E/19MAA/Na⁺(2.0 Ml)¹ | 72.45 | 61.38 |
| Hydroxystearic acid | 6.94 | 0 |
| Behenic acid | 0 | 15.34 |
| Potassium carbonate | 7.05 | 8.55 |
| E/24MA (20 Ml)² | 7.70 | 7.59 |
| E/2MAA/15iBA (130 Ml)³ | 5.88 | 7.14 |
| Film gauge | 2.4 mil | 2.7 mil |

Notes for Table 4:
¹Copolymer of ethylene with methacrylic acid (19 wt %) neutralized with sodium (37 mol % of acid groups) and having a melt index of 2.0.
²Copolymer of ethylene with methyl acrylate (24 wt %) having a melt index of 20.
³Copolymer of ethylene with methacrylic acid (2 wt %) and isobutyl acrylate (15 wt %), and having a melt index of 130.

Solutions of two antimicrobial agents, benzoic acid and nisin, were prepared, each at three concentrations. The composition of each antimicrobial solution is listed in Tables 5 and 6.

For each concentration of each antimicrobial agent, a spot on lawn assay was conducted as a positive control experiment to assess the efficacy of the antimicrobial solutions. In this assay, Trypticase Soy Agar (TSA) and Modified Oxford Agar (MOX) agar plates were inoculated with L. monocytogenes 15313 at a level of 10E+08 cfu/ml through an Autoplate 4000 model from Spiral Biotech, Inc., of Norwood, Mass. A volumetric pipette pump was then used to place three 10 μl spots of antimicrobial solution onto three well-separated regions of the test plate. Another volumetric pipette pump was used to place 10 μl of a negative control solution (i.e., neat solvent(s)) onto another well-separated region. Three such agar plates were prepared for each concentration of antimicrobial solution. After spot placement, the agar plates were allowed to stand undisturbed for 10 to 30 minutes in a laminar flow hood before incubation at 37° C. for at least 24 hours.

The zone of inhibition resulting from each spot of antimicrobial solution was measured using a dial caliper (available from Brown and Sharpe of North Kingstown, R.I.; accuracy to 0.02 mm). Each zone of inhibition was measured twice, once on each of two perpendicular axes. The values reported in Tables 2 (TSA plates) and 3 (MOX plates) are the average of 18 measurements. No zone of inhibition was observed for any of the control solutions.

In a film on lawn assay, films of Example 1 and Example 2 were cut into 15 mm by 15 mm samples, placed onto aluminum foil, and exposed to UV light in a laminar flow hood for 4 to 7 minutes. The samples were then transferred from the foil to one of six freshly made up antimicrobial solutions to imbibe for 12 to 24 hours at a specified temperature (4° C. or 37° C.). The films are expected to substantially reach equilibration in less than 12 hours. The films were removed with a pair of flame-sterilized ocular forceps, allowed to drip dry, and held for 60 seconds on a sterile Petri dish before positioning on TSA and MOX agar plates. The agar plates had been prepared as described above with respect to the spot on lawn assay. Two films were placed on each of the prepared agar plates, in well-separated regions. A third 15 mm×15 mm film was imbibed under the same conditions in a negative control solution and placed in another region of the plate. Three plates were prepared in this manner for each concentration of each antimicrobial agent. In the case of the MOX agar plates, a fourth plate was prepared with the same level of innoculum, with no films in it, to serve as a growth indicator plate for color change. After film placement, the plates were allowed to stand undisturbed for 10 to 30 minutes in a laminar flow hood before incubation for at least 24 hours.

Incubation took place at either 4° C. for 14 to 28 days or 37° C. for two to five days. The incubation conditions are noted in the Tables.

The zone of inhibition resulting from each spot of antimicrobial solution was measured as described above, with respect to the spot on lawn assay. The values reported in Tables 5 and 6 are the average of 12 measurements. Occasionally inhibition could be observed on the MOX agar plates, but not easily measured, owing to the relatively diffuse boundaries between areas of inhibition and non-inhibition. In such cases, a qualitative description of the results is provided. No zone of inhibition was observed for any of the control films.

TABLE 5
Results obtained from Assays on TSA Agar Plates

| | | Group averages of Inhibition Zones¹ (mm), TSA Agar | | |
|---|---|---|---|---|
| | | | Film on Lawn Assay | Film on Lawn Assay |
| | | | Example 1 | Example 2 |
| Antimicrobial Agent(s)⁰ | | Spot on | Film soaked at 37 C.; | Film soaked at 37 C.; |
| Component 1 | Solution² | Lawn Assay | Incubation at 37 C. | Incubation at 37 C. |
| Benzoic acid (0.75) | Ethanol/Water | 8.13 ± 0.27 | 0 (no inhibition) | 0 (no inhibition) |
| | (36/63.25) | | | |
| Benzoic acid (1.5) | Ethanol/Water | 8.32 ± 0.69 | 17.68 ± 1.64 | 18.60 ± 0.61 |
| | (36/62.5) | | | |
| Benzoic acid (3.0) | Ethanol/Water | 8.32 ± 0.53 | 20.17 ± 1.19 | 18.90 ± 1.82 |
| | (36/61) | | | |
| Nisin² (2500) | Water (100) | 9.94 ± 0.78 | 0 (no inhibition) | 0 (no inhibition) |
| Nisin² (5000) | Water (100) | 10.02 ± 0.41 | 0 (no inhibition) | 0 (no inhibition) |
| Nisin² (10000) | Water (100) | 9.37 ± 0.32 | 0 (no inhibition) | 0 (no inhibition) |

Notes for Table 5:
⁰The numbers in the parentheses are wt % (weight %), unless otherwise noted.
¹All control spots and control films showed no zones of inhibition.
²Nisin obtained from Sigma, a 2.5% concentration of nisin balanced with sodium chloride and denatured milk solids; the numbers in the parentheses are International Units per milliliter of solution (IU/ml).

TABLE 3
Results obtained from Assays on MOX Agar Plates

| Antimicrobial Agent(s)⁰ | | Group averages of Inhibition Zones¹ (mm), MOX Agar Plates | | | | |
|---|---|---|---|---|---|---|
| | | | Film on Lawn | Film on Lawn | Film on Lawn | Film on Lawn |
| | | | Assay | Assay | Assay | Assay |
| | | | Example 1 | Example 2 | Example 1 | Example 2 |
| | | Spot on | Film soaked at | Film soaked at | Film soaked at | Film soaked at |
| Component | | Lawn | 37 C.; Incubation | 37 C.; Incubation | 37 C.; Incubation | 37 C.; Incubation |
| 1 | Solution | Assay | at 37 C. | at 37 C. | at 4 C. | at 4 C. |
| Benzoic acid | Ethanol/ | 7.91 ± | 0 (no inhibition) | 0 (no inhibition) | | |
| (0.75) | Water | 0.77 | | | | |
| | (36/63.25) | | | | | |
| Benzoic acid | Ethanol/ | 8.66 ± | 24.0 ± 3.34 | 21.38 ± 3.90 | | |
| (1.5) | Water | 0.66 | | | | |
| | (36/62.5) | | | | | |
| Benzoic acid | Ethanol/ | 8.75 ± | 39.52 ± 2.93 | 32.29 ± 7.53 | | |
| (3.0) | Water | 0.80 | | | | |
| | (36/61) | | | | | |
| Nisin² | Water | 24.99 ± | No inhibition to | No inhibition to | 40.08 ± 3.42 | 33.90 ± 2.89 |
| (2500) | (100) | 1.30 | moderate | moderate | | |
| | | | inhibition | inhibition | | |
| Nisin² | Water | 25.89 ± | No inhibition to | No inhibition to | 36.97 ± 5.35 | 40.95 ± 5.55 |
| (5000) | (100) | 1.59 | distinct inhibition | moderate | | |
| | | | | inhibition | | |
| Nisin² | Water | 28.80 ± | Clear zone | No inhibition to | 41.57 ± 4.94 | 40.23 ± 3.43 |
| (10000) | (100) | 2.45 | around film to | distinct inhibition | | |
| | | | distinct inhibition | | | |
| | | | Film on Lawn | Film on Lawn | | |
| | | | Assay | Assay | | |
| | | | Example 1 | Example 2 | | |
| | | | Film soaked at | Film soaked at | | |
| | | | 4 C.; Incubation | 4 C.; Incubation | | |
| | | | at 37 C. | at 37 C. | | |
| Nisin² | Water | | 22.14 ± 1.24 | | | |
| (25000) | (100) | | | | | |
| Nisin² | Water | | 22.70 ± 1.22 | 25.98 ± 1.42 | | |
| (50000) | (100) | | | | | |
| Nisin² | Water | | 23.43 ± 0.75 | 27.23 ± 1.56 | | |
| (100000) | (100) | | | | | |

Notes for Table 6:
⁰The numbers in the parentheses are wt % (weight %), unless otherwise noted.
¹All control spots and control films showed no zones of inhibition.
²Nisin obtained from Sigma, a 2.5% concentration of nisin balanced with sodium chloride and denatured milk solids; the numbers in the parentheses are International Units per milliliter of solution (IU/ml).

While certain of the preferred embodiments of the present invention have been described and specifically exemplified above, it is not intended that the invention be limited to such embodiments. Various modifications may be made without departing from the scope and spirit of the present invention, as set forth in the following claims.

What is claimed is:

1. A controlled release antimicrobial polymer composition comprising:
at least one E/X/Y copolymer, or at least one ionomer of an E/X/Y copolymer, wherein E is ethylene, X is a C₃ to C₈ α,β-ethylenically unsaturated carboxylic acid, and Y is a softening comonomer selected from alkyl acrylates and alkyl methacrylates, wherein the alkyl groups have from one to eight carbon atoms, and wherein the amount of X ranges from about 0.1 to about 40 weight % of the E/X/Y copolymer or ionomer, and wherein the amount of Y ranges from 0 to about 40 weight % of the E/X/Y copolymer or ionomer;
at least one organic acid, wherein the acid comprises the acid or one or more salts thereof, or a mixture of the acid and one or more salts thereof; and
at least one antimicrobial agent.

2. The controlled release antimicrobial polymer composition of claim 1, wherein the amount of X ranges from about 5 to about 30 wt % of the E/X/Y ionomer, and the amount of Y ranges from 0 to about 30 wt % of the E/X/Y ionomer.

3. The controlled release antimicrobial polymer composition of claim 1, wherein the carboxylic acid groups of X and of the organic acid are neutralized to a level of 15 mol % to 100 mol % with monovalent or divalent metal ions.

4. The controlled release antimicrobial polymer composition of claim 3, wherein the monovalent or divalent metal ions comprise one or more alkali metal ions.

5. The controlled release antimicrobial polymer composition of claim 3, wherein the monovalent or divalent metal ions comprise potassium cations.

6. The controlled release antimicrobial polymer composition of claim 1, wherein the at least one E/X/Y copolymer, or the at least one ionomer of an E/X/Y copolymer, is present in an amount of from about 1 to about 100 wt %, based on the total weight of the controlled release antimicrobial polymer composition.

7. The controlled release antimicrobial polymer composition of claim 1, wherein the at least one organic acid, at least one organic acid salt, or combination of least one organic acid and at least one organic acid salt are selected from the group consisting of caproic acid, caprylic acid, capric acid, lauric acid, myristic acid, palmitic acid, stearic acid, hydroxystearic acid, dihydroxystearic acid, oleic acid, linoleic acid, alpha-linolenic acid, dihomo-gamma-linolenic acid, erucic acid, butyric acid, arachidic acid, arachidonic acid, behenic acid, lignoceric acid, cerotic acid, lauroleic acid, myristoleic acid, pentadecanoic acid, palmitoleic acid, margaric acid, ricinoleic acid, elaidic acid, eleostearic acid, licanic acid, eicosenoic acid, eicosanoic acid, eicosapentaenoic acid, docosahexaenoic acid, montanic acid, citric acid; the salts thereof; and the isomers thereof.

8. The controlled release antimicrobial polymer composition of claim 6, wherein the at least one organic acid, at least one organic acid salt, or combination of at least one organic acid and at least one organic acid salt are selected from the group consisting of oleic acid, stearic acid, behenic acid, hydroxystearic acid, the salts thereof, and the isomers thereof.

9. The controlled release antimicrobial polymer composition of claim 1, wherein the at least one organic acid, at least one organic acid salt, or combination of least one organic acid and at least one organic acid salt is present in an amount of from about 0.1 wt % to about 50 wt %, based on the total weight of the controlled release antimicrobial polymer composition.

10. The controlled release antimicrobial polymer composition of claim 1, wherein the at least one organic acid, at least one organic acid salt, or combination of least one organic acid and at least one organic acid salt is present in an amount of from about 5 wt % to about 20 wt %, based on the total weight of the controlled release antimicrobial polymer composition.

11. The controlled release antimicrobial polymer composition of claim 1, wherein the antimicrobial agent is selected from the group consisting of water soluble alcohols; water miscible alcohols; phenolic compounds; benzoic acid and its salts; sorbic acid and its salts; metal containing compositions; quaternary ammonium compounds; biguanides; bis-biguanide alkanes; short chain alkyl esters of p-hydroxybenzoic acid, commonly known as parabens; N-(4-chlorophenyl)-N′-(3,4-dichlorophenyl) urea; azoles; chitosan; and derivatives of tetracycline, thienamycin, chloramphenicol, cefoxitin, neomycin, fluoroquinolone, fatty acid salts, sulfonamides, and aminoglycoside that have hydrophilic solvent or water solubility; and combinations of two or more thereof.

12. The controlled release antimicrobial polymer composition of claim 11, wherein the antimicrobial agent comprises a metal containing composition comprising at least one metal selected from the group consisting of silver, gold, copper, zinc, mercury, tin, lead, bismuth, cadmium, chromium and thallium.

13. The controlled release antimicrobial polymer composition of claim 11, wherein the antimicrobial agent comprises a metal containing composition comprising silver.

14. The controlled release antimicrobial polymer composition of claim 1, wherein the antimicrobial agent is present in an amount of from about 0.0000001 wt % to about 10 wt %, based on the total weight of the controlled release antimicrobial polymer composition.

15. The controlled release antimicrobial polymer composition of claim 1, wherein the antimicrobial agent comprises silver, and wherein the silver is present in an amount of from about 0.0005 wt % to about 5 wt %, based on the total weight of the controlled release antimicrobial polymer composition.

16. An article comprising the controlled release antimicrobial polymer composition of claim 1.

17. The article of claim 16, being a pellet, granule, powder, strand, or semi-finished shape.

18. An article comprising the pellet, granule, powder, strand, or semi-finished shape of claim 17.

19. The article of claim 16, being a medical device.

20. The medical device of claim 19, wherein the antimicrobial agent comprises silver.

21. The medical device of claim 20, being a catheter.

22. A controlled release antimicrobial polymer composition comprising
at least one ionomer of an E/X/Y copolymer, wherein E is ethylene, X is a C₃ to C₈ α,β-ethylenically unsaturated carboxylic acid, and Y is a softening comonomer selected from alkyl acrylates and alkyl methacrylates; wherein the alkyl groups have from one to eight carbon atoms; wherein the amount of X ranges from about 0.1 to about 40 wt % of the E/X/Y copolymer, and the amount of Y ranges from 0 to about 40 weight % of the E/X/Y copolymer; and wherein the at least one ionomer is at least partially neutralized with one or more monovalent cations; and
at least one antimicrobial agent.

23. The controlled release antimicrobial polymer composition of claim 22, wherein the amount of X ranges from about 5 wt % to about 30 wt % of the E/X/Y ionomer; the amount of Y ranges from 0 to about 30 wt % of the E/X/Y ionomer.

24. The controlled release antimicrobial polymer composition of claim 22, wherein the carboxylic acid groups of X are neutralized to a level of 15 mol % to 100 mol % with the one or more monovalent cations.

25. The controlled release antimicrobial polymer composition of claim 22, wherein the one or more monovalent cations comprise one or more alkali metal cations.

26. The controlled release antimicrobial polymer composition of claim 22, wherein the one or more monovalent cations comprise potassium ions.

27. The controlled release antimicrobial polymer composition of claim 26, wherein the one or more monovalent cations comprise potassium ions.

28. The controlled release antimicrobial polymer composition of claim 22, wherein the antimicrobial agent is selected from the group consisting of water soluble alcohols; water miscible alcohols; phenolic compounds; benzoic acid and its salts; sorbic acid and its salts; metal containing compositions; quaternary ammonium compounds; biguanides; bis-biguanide alkanes; short chain alkyl esters of p-hydroxybenzoic acid, commonly known as parabens; N-(4-chlorophenyl)-N′-(3,4-dichlorophenyl) urea; azoles; chitosan; and derivatives of tetracycline, thienamycin, chloramphenicol, cefoxitin, neomycin, fluoroquinolone, fatty acid salts, sulfonamides, and aminoglycoside that have hydrophilic solvent or water solubility; and combinations of two or more thereof.

29. The controlled release antimicrobial polymer composition of claim 28, wherein the antimicrobial agent is a metal containing composition comprising at least one metal selected from the group consisting of silver, gold, copper, zinc, mercury, tin, lead, bismuth, cadmium, chromium and thallium.

30. The controlled release antimicrobial polymer composition of claim 28, wherein the antimicrobial agent comprises silver.

31. The controlled release antimicrobial polymer composition of claim 22, wherein the antimicrobial agent is present in an amount of from about 0.0000001 to about 10 wt %, based on the total weight of the controlled release antimicrobial polymer composition.

32. The controlled release antimicrobial polymer composition of claim 22, wherein the antimicrobial agent comprises silver, and wherein the silver is present in an amount of from about 0.0005 wt % to about 5 wt %, based on the total weight of the controlled release antimicrobial polymer composition.

33. The controlled release antimicrobial polymer composition of claim 22, further comprising at least one organic acid, wherein the acid comprises the acid or one or more salts thereof, or a mixture of the acid and one or more salts thereof.

34. The controlled release antimicrobial polymer composition of claim 33, wherein the at least one organic acid is selected from the group consisting of caproic acid, caprylic acid, capric acid, lauric acid, myristic acid, palmitic acid, stearic acid, hydroxystearic acid, dihydroxystearic acid, oleic acid, linoleic acid, alpha-linolenic acid, dihomo-gamma-linolenic acid, erucic acid, behenic acid, butyric acid, arachidic acid, arachidonic acid, lignoceric acid, cerotic acid, lauroleic acid, myristoleic acid, pentadecanoic acid, palmitoleic acid, margaric acid, ricinoleic acid, elaidic acid, eleostearic acid, licanic acid, eicosenoic acid, eicosapentaenoic acid, docosahexaenoic acid, montanic acid, citric acid; the salts thereof; and the isomers thereof.

35. The controlled release antimicrobial polymer composition of claim 33, wherein the at least one organic acid is selected from the group consisting of oleic acid, stearic acid, behenic acid, hydroxystearic acid, the salts thereof, and the isomers thereof.

36. The controlled release antimicrobial polymer composition of claim 33, wherein the at least one organic acid is present in an amount of from about 0.1 to about 50 wt %, based on the total weight of the controlled release antimicrobial polymer composition.

37. The controlled release antimicrobial polymer composition of claim 33, wherein the at least one organic acid is present in an amount of from about 5 wt % to about 20 wt %, based on the total weight of the controlled release antimicrobial polymer composition.

38. An article comprising the controlled release antimicrobial polymer composition of claim 22.

39. The article of claim 38, being a pellet, granule, powder, strand, or semi-finished shape.

40. An article comprising the pellet, granule, powder, strand, or semi-finished shape of claim 39.

41. The article of claim 38, being a medical device.

42. The medical device of claim 41, wherein the antimicrobial agent comprises silver.

43. The medical device of claim 42, being a catheter.

44. A method of producing a controlled release antimicrobial polymer composition, comprising
providing a polymer composition comprising at least one E/X/Y acid copolymer or at least one ionomer of an E/X/Y acid copolymer, wherein E is ethylene, X is a C₃ to C₈ α,β-ethylenically unsaturated carboxylic acid, and Y is a softening comonomer selected from alkyl acrylates and alkyl methacrylates; wherein the alkyl groups have from one to eight carbon atoms; and wherein the amount of X ranges from about 0.1 to about 40 weight % of the E/X/Y copolymer, and the amount of Y ranges from 0 to about 40 weight % of the E/X/Y copolymer; and wherein the at least one ionomer is at least partially neutralized with monovalent cations; and
incorporating an antimicrobial agent into the polymer composition; and, optionally,
further neutralizing the polymer composition with a neutralizing agent.

45. The method of claim 44, wherein the polymer composition further comprises at least one organic acid, wherein the acid comprises the acid or one or more salts thereof, or a mixture of the acid and one or more salts thereof.

46. The method of claim 44, wherein the antimicrobial agent is incorporated into the polymer blend by melt blending.

47. The method of claim 44, wherein the polymer composition comprises at least one ionomer of an E/X/Y acid copolymer, and wherein the antimicrobial agent is incorporated into the polymer blend by contacting the polymer composition or the polymer blend with a liquid comprising the antimicrobial agent.

48. The method of claim 47, wherein the liquid comprising the antimicrobial agent further comprises water.

49. The method of claim 48, wherein the antimicrobial agent is selected from the group consisting of water soluble alcohols; water miscible alcohols; phenolic compounds; benzoic acid and its salts; sorbic acid and its salts; metal containing compositions; quaternary ammonium compounds; biguanides; bis-biguanide alkanes; short chain alkyl esters of p-hydroxybenzoic acid; N-(4-chlorophenyl)-N′-(3,4-dichlorophenyl) urea; azoles; and derivatives of tetracycline, thienamycin, chloramphenicol, cefoxitin, neomycin, fluoroquinolone, fatty acid salts, sulfonamides, and aminoglycoside that have hydrophilic solvent or water solubility; and combinations of two or more thereof.

50. The method of claim 49, wherein the antimicrobial agent comprises a metal containing composition comprising at least one metal selected from the group consisting of silver, gold, copper, zinc, mercury, tin, lead, bismuth, cadmium, chromium and thallium.

51. The method of claim 50, wherein the antimicrobial agent comprises one or more metal ions.

52. The method of claim 51, wherein the antimicrobial agent comprises silver.

53. The method of claim 47, wherein the liquid comprising the antimicrobial agent is sprayed onto the polymer composition.

54. The method of claim 53, wherein the liquid is heated.

55. The method of claim 47, wherein the polymer composition is at least partially immersed in the liquid comprising the antimicrobial agent.

56. The method of claim 55, wherein the liquid is heated.

57. The method of claim 47, comprising two or more contacting steps, which may be the same or different.

58. The method of claim 57, wherein a first antimicrobial agent is incorporated into the polymer composition by melt blending, to produce a resulting polymer composition, and wherein a second antimicrobial agent is incorporated into the resulting polymer composition by contacting the resulting polymer composition with a liquid comprising the second antimicrobial agent, and wherein the first antimicrobial agent and the second antimicrobial agent may be the same or different.

59. A pellet, granule, powder, strand, or semi-finished shape obtainable by the process of claim 44.

60. An article comprising the pellet, granule, powder, strand, or semi-finished shape of claim 59.

61. An article comprising the controlled release antimicrobial polymer obtainable by the method of claim 44.

62. The article of claim 61 being a medical device.

63. The medical device of claim 62 being a catheter.

64. A method of providing a polymer blend with antimicrobial properties, comprising
providing a polymer blend comprising a polymer composition comprising at least one ionomer of an E/X/Y copolymer, wherein E is ethylene, X is a C₃ to C₈ α,β-ethylenically unsaturated carboxylic acid, and Y is a softening comonomer selected from alkyl acrylates and alkyl methacrylates; wherein the alkyl groups have from one to eight carbon atoms; and wherein the amount of X ranges from about 0.1 to about 40 weight % of the E/X/Y copolymer, and the amount of Y ranges from 0 to about 40 weight % of the E/X/Y copolymer; and wherein the at least one ionomer is at least partially neutralized with monovalent cations; and
incorporating an antimicrobial agent into the polymer blend; and, optionally,
further neutralizing the polymer composition with a neutralizing agent.

65. A method of providing an article with antimicrobial properties, comprising
providing an article comprising a polymer composition; the polymer composition comprising at least one ionomer of an E/X/Y copolymer, wherein E is ethylene, X is a C₃ to C₈ α,β-ethylenically unsaturated carboxylic acid, and Y is a softening comonomer selected from alkyl acrylates and alkyl methacrylates; wherein the alkyl groups have from one to eight carbon atoms; and wherein the amount of X ranges from about 0.1 to about 40 weight % of the E/X/Y copolymer, and the amount of Y ranges from 0 to about 40 weight % of the E/X/Y copolymer; and
contacting the article with a liquid comprising an antimicrobial agent.
